(19) United States
(12) Patent Application Publication
(10) Pub. No.: US 2007/0202155 A1
(43) Pub. Date: Aug. 30, 2007

(54) Low dose no donor-containing transdermal patch

(75) Inventors: Robert Ang, New York, NY (US); Ludwig Weimann, San Diego, CA (US); Sven Jacobson, New York, NY (US)

Correspondence Address: THORPE NORTH & WESTERN, LLP., 8180 SOUTH 700 EAST, SUITE 350, SANDY, UT, 84070, US

(73) Assignee: CURE THERAPEUTICS, INC.

(21) Appl. No.: 11/701,964

(22) Filed: Feb. 1, 2007

Related U.S. Application Data
(60) Provisional application No. 60/765,253, filed on Feb. 3, 2006.

(51) Int. Cl. A61K 9/70; A61K 31/21

(52) U.S. Cl. 424/449; 514/509

(57) ABSTRACT

The present invention is drawn to a transdermal patch for the delivery of a nitroglycerin or other NO donor. The patch can comprise a backing layer, and a nitroglycerin-containing composition or other NO donor-containing composition which is supported at least in part by the backing layer. The transdermal patch can have a drug delivery zone defined by the area where the composition contacts an intact human skin site, and the transdermal patch can be formulated to deliver a nitric oxide donor, such as nitroglycerin, at from about 5 μg/hour to about 70 μg/hour. In one embodiment, the transdermal patch can provide a delivery rate at the drug delivery zone of from about 1 μg/cm²/day to about 600 μg/cm²/day. In another embodiment, the transdermal patch can contain from 6 wt % to 18 wt % of the nitric oxide donor, e.g., nitroglycerin. In each embodiment, the patch can contain at least 450 μg/cm² of nitroglycerin.

This application claims the benefit of U.S. Provisional Application No. 60/765,253, filed on Feb. 3, 2006.

### FIELD OF THE INVENTION

The present invention relates to devices for delivering nitric oxide to treat various tissue sites. In particular the present invention includes a low dose nitroglycerin transdermal patch.

### BACKGROUND OF THE INVENTION

Nitric Oxide (NO) is a highly reactive chemical species and is extremely short-lived, so NO is most typically supplied to a patient in the form of glyceryl trinitrate (GTN, also known as nitroglycerin) or through some other substance capable of generating NO (termed “nitric oxide donor”). Such donor substances tend to be more stable than NO itself and can thereby be used to release NO over time. NO donors can be administered sublingually (e.g., by tablets placed under the tongue), transdermally (e.g., by a dermal composition placed on the skin), or in other ways.

NO is produced endogenously by three isoforms of the enzyme nitric oxide synthase, inducible nitric oxide synthase (iNOS), an isoform originally found in endothelial cells (eNOS), and an isoform originally found in brain tissue and neuronal cells (bNOS). NO is produced in large amounts by inflammatory cells such as macrophages, neutrophils, lymphocytes, and peripheral-blood monocytes during immunological reactions and septic shock. There is also an inducible form of nitric oxide synthase in cartilage.

NO is believed to act as a vasodilator and has been found to be useful in treating several disorders, most notably angina pectoris. NO has also been shown to provide enhanced or accelerated wound healing and relief of pain. Wound healing involves the recruitment of inflammatory cells, followed by fibroblasts, to the site of the wound, where collagen and other connective tissue elements are deposited. The collagen fibers then gradually realign to resemble the original connective tissue (e.g., tendon, ligament, skin, etc.). Topical NO donation has been used effectively to treat cutaneous wounds and tendons in animal models via mechanisms that may include stimulation of collagen synthesis in fibroblasts.

Because NO has been shown to be effective in treating a wide variety of ailments and conditions, researchers continue to make efforts to develop new NO formulations for providing effective NO delivery.

### SUMMARY OF THE INVENTION

The present invention is drawn to methods and devices for use in treating subjects with nitric oxide donor-containing compositions, in particular with nitroglycerin containing compositions. In a first embodiment, a transdermal patch for the delivery of a nitroglycerin can comprise a backing layer, and a nitroglycerin-containing composition supported at least in part by the backing layer. The transdermal patch can have a drug delivery zone defined by the area where the composition contacts an intact human skin site, and the drug delivery zone can have an area of at least 2.5 cm². The transdermal patch can be formulated to deliver nitroglycerin at from about 5 μg/hour to about 70 μg/hour, and wherein the nitroglycerin-containing composition comprises from 6.0% wt % to 18.0 wt % nitroglycerin. The patch should have a minimum concentration of nitroglycerin of at least 450 μg/cm².

In another embodiment, a transdermal patch for the delivery of a nitric oxide donor can comprise a backing layer, and a nitric oxide donor-containing composition comprising at least one nitric oxide donor other than nitroglycerin which is supported at least in part-by the backing layer. The transdermal patch can have a drug delivery zone-defined by the area where the composition contacts an intact human skin site, and the transdermal patch can be formulated to deliver the nitric oxide donor other than nitroglycerin at from about 5 μg/hour to about 70 μg/hour. Further, the nitric oxide donor-containing composition comprises from 6.0 wt % to 18.0 wt % nitroglycerin. The patch should have a minimum concentration of nitroglycerin of at least 450 μg/cm².

In another embodiment, a method for delivering a nitric oxide donor to a subject in need thereof can comprise the step of applying a nitric oxide donor-containing transdermal patch to a skin surface, said transdermal patch including a nitric oxide donor-containing composition and having a drug delivery zone defined by the area where the composition is contacted by intact human skin site. The transdermal patch can be formulated to deliver from about 5 μg/hour to about 70 μg/hour, and further, the nitric oxide donor containing composition comprises from 6.0 wt % to 18.0 wt % of a nitric oxide donor. The patch should have a minimum concentration of nitric oxide donor of at least 450 μg/cm².

In still another embodiment, a transdermal matrix patch for delivering nitroglycerin can comprise a backing layer and a nitroglycerin-containing matrix layer. The nitroglycerin-containing matrix layer can be supported at least in part by the backing layer. The transdermal matrix patch can have a drug delivery zone defined by the area where the nitroglycerin-containing matrix layer contacts an intact human skin site and can have a delivery rate of from about 5 μg/hour to about 70 μg/hour. Further the transdermal patch can also provide a delivery rate at the drug delivery zone from about 1 μg/cm²/day to about 600 μg/cm²/day, with the proviso that the patch contains at least 450 μg/cm² of nitroglycerin.

In another embodiment, a transdermal matrix patch for delivering a nitric oxide donor can comprise a backing layer and a nitric oxide donor-containing matrix layer. The nitric oxide donor-containing matrix layer can be supported at least in part by the backing layer. The transdermal matrix patch can have a drug delivery zone defined by the area where the nitric oxide donor-containing matrix layer contacts an intact human skin site and can have a delivery rate of from about 5 μg/hour to about 70 μg/hour. Further the transdermal patch can also provide a delivery rate at the drug delivery zone from about 1 μg/cm²/day to about 600 μg/cm²/day, with the proviso that the patch contains at least 450 μg/cm² of the nitric oxide donor.

Exemplary subjects in need thereof i) may be experiencing pain and the transdermal patch is applied for reducing this pain, ii) may have damaged tissue and the transdermal patch is applied for accelerating healing of the damaged tissue, iii) may have tendinopathy (tendinosis or tendonitis) and the transdermal patch is applied to improve function of an afflicted tendon iv) may have cancer, v) may be suffering from acute inflammation, and/or vi) may have other physical ailments.

### DETAILED DESCRIPTION OF THE PREFERRED EMBODIMENT(S)

Before particular embodiments of the present invention are disclosed and described, it is to be understood that this invention is not limited to the particular process and materials disclosed herein and as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and, “the” include plural referents unless the context clearly dictates otherwise. Thus, for example, reference to a drug-containing composition including “an NO-donor” includes one or more NO-donors and reference to “the nitric oxide donor” includes reference to one or more nitric oxide donors.

As used herein, “subject” refers to a mammal that may benefit from the administration of the systems or methods of this invention. Examples of subjects include humans, and may also include other animals such as horses, pigs, cattle, dogs, cats, rabbits, and aquatic mammals.

As used herein, the terms “formulation” and “composition” are used interchangeably and refer to mixtures, solutions, dispersions, etc. of two or more compounds, elements, or molecules.

As used herein, the term “nitric oxide donor” or “NO-donor” refers a compound or mixture of compounds which, when delivered to a subject, increases the concentration of nitric oxide present in the subject. In some embodiments, the nitric oxide donor includes nitroglycerin and in other embodiments, nitroglycerin is specifically excluded.

As used herein, the term “drug delivery zone” refers to the area of skin which comes into direct contact with the portion of the transdermal patch which delivers the nitric oxide donor. For example, when the delivery device is a transdermal matrix patch the drug delivery zone would be the area in which the drug containing matrix contacts the skin.

As some NO-donors may cause undesirable tolerance issues, in some cases it can be desirable allow for drug holidays. “Drug holiday(s)” refer to periods of time in which the transdermal patch is removed for a predetermined length of time before a subsequent patch is administered. For example, a patch of the present invention may be applied to the skin of a subject for a period of 12 hours after which the patch is removed and a drug holiday period of 12 hours is allowed to pass before a subsequent patch is applied to the subject's skin. Other periods of time for drug delivery and drug holidays can also be implemented, as would be known to those skilled in the art after considering the present disclosure.

As used herein the term “continuous” or “continuously” in the context of drug administration refers to regular or constant dosing of predetermined amounts of a NO donor to a subject. For the purposes of the present invention the incorporation of planned regular drug holidays into a dosing regimen does not destroy the continuous nature of the drug administration period. Continuous drug administration also covers periods where a new transdermal patch is applied directly after the removal of a used patch without a drug holiday.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

NO can be locally delivered to and beneficial to a site for a variety of reasons including providing pain relief, improving wound healing, improving function, reducing inflammation, treating or preventing angina, inhibiting cancer metastasis, etc. The local delivery can be accomplished through the use of a transdermal patch which is placed on a skin surface proximate, distal, or over the painful or wounded site. As nitroglycerin, a preferred NO donor, is typically rapidly systemically absorbed and distributed, it is feasible that a patch of the present invention could be placed on a skin site which is remote from the site requiring treatment and still be effective for providing the desired outcome. Preferably, the transdermal patch is applied proximate the area for which pain relief, wound healing, or improved function is desired, but this is not required. To provide one example, NO delivery in accordance with embodiments of the present invention can be carried out to treat tendons suffering from tendinopathy, including either tendinosis or tendonitis.

For example, if an Achilles tendon is in need of pain relief, wound healing, or other treatment, a transdermal patch containing an NO donor-containing composition may be placed on the skin proximate the Achilles tendon. In this embodiment, the patch releases the NO donor and the increased NO concentrations can provide for reduced pain, accelerated wound healing, and/or improved function. Other examples of uses for the transdermal patch of the present invention include but are not limited to the enhancing healing of damaged muscles or reducing the pain associated therewith, enhancing healing and relieving pain of a chronic of a chronic skin ulcer, treatment of heart failure, treatment of angina, tendonitis, tendinosis, prevention of cancer metastasis, prevention of thrombophlebitis, treatment of acute inflammation including inflammation associated with acute local arthritis and thrombophlebitis, accelerate bone healing, accelerate post-operative recovery, prevention of unnecessary inflammation, etc.

There are significant advantages of applying an NO donor transdermal patch to a skin site rather than a cream or ointment. Perhaps the greatest advantage is that transdermal patches can provide measured sustained release of a drug over a desired period of time. Another benefit is that a patch can be easily removed in the event that a patient experiences unwanted side-effects associated with the patch. A further advantage of using a transdermal patch relates to the ability to deliver more precise dosages. Although creams and ointments can be prepared to contain specific concentrations of an NO donor, they can be applied at any thickness and over any area of skin yielding inconsistent dosing. This inconsistent dosing can lead to over dosing or under dosing of the NO donor. Overdosing can result in unwanted side effects including severe skin irritation, headaches, or vascular problems including hypotension. Under dosing can result in ineffective treatment.

It has been discovered that by more precise dosing to the tissue using a low dose patch over a larger surface area, irritation can be reduced, skin patch adherence is improved, and appropriate amounts of NO donor can be delivered over a larger surface area where needed. Larger areas of attachment, or larger drug delivery zones, also have the added benefit when the application site is to a mobile joint, e.g., elbow, knee ankle, etc, or an area in which large amounts of rubbing or contact can occur e.g. back, legs, arms, etc. As afflicted areas are often joints, the NO-donor containing patch may be of a size that makes it easy to apply and remove when desired, and further, the patch can be manufactured to be a size that is more likely to stay affixed to a joint while still delivering low doses of an NO-donor. In other words, it has been recognized that more precise low doses can treat various tissue ailments and provide the significant advantages associated with delivering these low dosages over drug delivery zones that are larger in area per dosage delivered than those previously known.

Certain NO-donor containing transdermal patches, such as nitroglycerin patches, already exist and can in theory be used to provide local NO therapy for pain relief and wound healing. There are, however, several disadvantages to such an approach. Commercially available nitroglycerin patches are designed for treating angina pectoris and are therefore formulated to release a dose appropriate for this use, generally a substantially higher dosage amount than necessary to provide other desirable affects such as wound healing, pain relief, or improved function. The minimum recommended dose for angina treatment is 0.2 mg/hour, or 5.0 mg/day, although a smaller 0.1 mg/hour patch, or 2.5 mg/day is also available but is generally intended to supplement the larger 5.0 mg/day patches. A single nitroglycerin patch typically contains enough nitroglycerin to release at the indicated rate per hour over the course of one day, so a nitroglycerin patch normally includes at least 20 mg nitroglycerin per patch. As this amount provides a dose which greatly exceeds that which is necessary to achieve beneficial results such as accelerate wound healing, pain relief, improved function, and/or other ailments, use of such a high-dose nitroglycerin patch for these purposes can result in unwanted side effects, such as hypotension, headaches, cardiac arrhythmias, flushing, dizziness, etc.

If the desire is to use commercially available patches for delivering lower dosages, these patches can be cut into smaller sizes in order to reduce the unwanted side effects mentioned above. For example, a standard 5 mg/day patch must be cut into quarters to deliver 1.25 mg/day, a dosage amount which may be appropriate for pain relief and wound healing. This results in a small patch that can be cumbersome to apply and remove, and further, may not cover the entire site to be treated, thereby limiting the intended pain relief and wound healing. Further, because of the small size of the modified patch, it can be more susceptible to inadvertent removal. For example, the skin sites that are proximate to the tissue areas most frequently in need of treatment are generally subject to large amounts of stretching, e.g. joints. As commercially available patches are designed to be placed on regions of skin that do not bend or stretch excessively, such as on the chest or upper arms, placement instructions for these patches typically specify that a patch is not to be placed near an elbow, knee, or other joint, which are all common areas in need of pain relief or wound healing. This is because joint motion can cause the patch to work free from the skin thereby reducing or eliminating the delivery of nitroglycerin.

Commercially available patches are not designed to be placed in areas with high skin stretch and rubbing, such as ankles, elbows, or exposed joints, therefore the adhesives used in these patches have relatively mild adhesion. The use of these lower stick adhesives is another factor which allows for the patches to become partially or completely dislodged from the skin thereby reducing or eliminating delivery of nitroglycerin. Thus, the combination of lower stick adhesives over a small surface area provides a combination of physical characteristics that is often unacceptable for application to joints. It is notable that some NO donors which can be used in the present invention, in particular nitroglycerin, are excellent plasticizers and can enhance the adhesive properties of an adhesive matrix patch. For example, incorporation of high concentrations of nitroglycerin into the adhesive matrix can allow for the use of adhesives that have milder adhesive properties. Stickier adhesives may be desirable if the concentrations of nitroglycerin or other NO donor are low.

In accordance with embodiments of the present invention, is has been recognized that low dose transdermal patches that overcome these disadvantages can be prepared to deliver low dosages of NO donors over a larger skin contact zone, or drug delivery zone, than has been previously delivered. The transdermal patches of the present invention can deliver a dose of nitroglycerin or other NO donor that is pharmaceutically effective for promoting beneficial results, such as pain relief, wound healing, improving function, while reducing or eliminating the risks and inconveniences of traditional nitroglycerin patches. The patches of the present invention can be sized for convenient application and removal to all areas of the body, but in particular to areas where there are large amounts of skin stretch or rubbing. For all sizes of patches of the present invention, the minimum delivery zone size is 2.5 cm². In a preferred embodiment the drug delivery zone can be at least 5 cm².

There are a wide variety of nitric oxide donors which can be used in the present invention. Examples of nitric oxide donors include but are not limited to nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof. In a preferred embodiment, the nitric oxide donor is nitroglycerin.

As mentioned, a preferred NO donor is nitroglycerin or glyceryl trinitrate (GTN, also called 1,2,3-propanetriol trinitrate). Nitroglycerin is exemplary of one preferred NO-donor for use in accordance with embodiments of the present invention. The transdermal patch of the present invention can provide for reduced pain, wound healing, or improved function by delivering the nitric oxide donor at dosage rates of from about 5 μg/hour to about 70 μg/hour, a range which is lower than the lowest dosage of 0.1 mg/hour currently commercially available for use in treating angina. Further, in one embodiment, the NO-donor release rate can be from about 10 μg/hour to about 60 μg/hour.

A unique feature of the present invention is that the patches have a reduced delivery rate per cm² of the drug delivery zone. The reduced delivery rate per cm² of the drug delivery zone allows for a patch with a relatively large drug delivery zone to deliver relatively small amounts of nitroglycerine or other NO-donor. As noted above, present nitroglycerin angina patches have to be cut into small pieces in order to achieve the drug dosage amounts used in accordance with embodiments of the present invention. When cut, these modified patches can deliver low dosages of nitroglycerin, however, these patches do not approach the required rate per cm² of the drug delivery zone as set forth herein. As mentioned previously, the cutting of larger patches to achieve low dose delivery can result in patch pieces that are difficult to handle, apply, and maintain in place. Further, skin irritation is more likely at the site of application, as the delivery rate at the smaller site is greater than the delivery rate per cm² when using transdermal patches prepared in accordance with embodiments of the present invention.

This being stated, in one embodiment, the patches of the present invention can provide a delivery rate of the nitric oxide donor to the drug delivery zone in amounts of from about 1 μg/cm²/day to about 600 μg/cm²/day. In another embodiment, the patches can deliver from about 1 μg/cm²/day to about 280 μg/cm²/day. In yet another embodiment the patches can deliver from 10 μg/cm²/day to about 280 μg/cm²/day. In a further embodiment the patches can deliver from about 50 μg/cm²/day to about 250 μg/cm²/day. The lower delivery rates of the nitric oxide donor allow for the increase in the patch's drug delivery zone without increasing the dosage amount delivered to the patient. The lower dosage amounts can also decrease or eliminate some of the side effects which are affiliated with high dosages of nitroglycerin, namely headache, lightheadedness, and hypotension. The patches of the present invention contain at least 450 μg/cm² of nitroglycerin.

As described above, each patch of the present invention has an area, known as the drug delivery zone, which is defined to be the area where the nitric oxide donor-containing composition contacts an intact human skin surface. The area of the drug delivery zone can vary depending on the desired rate of delivery per cm² of the drug delivery zone and the total dosage amount to be delivered in a given dosing period. The size of the drug delivery zone can be from about 2.5 cm² to about 100 cm². In another embodiment, the size of the drug delivery zone is from about 3 cm² to about 50 cm².

The patches of the present invention can be used for administering NO-donors for both short and long periods of time. For reduced pain, wound healing, and improved function, the transdermal patch can be formulated to be able to sustain delivery of nitroglycerin (or other NO donor) over a continuous period of time of from 4 hours to 7 days. In another embodiment, the transdermal patch is formulated to deliver the nitric oxide donor for a continuous period of from about 1 to about 3 days. In a further embodiment the transdermal patch is formulated to deliver the nitric oxide donor for a continuous period of from about 12 hours to 24 hours. Patches can also be continuously administered over an administration period of from about 1 week to about 1 year. In one embodiment, a continuous administration period can last from about 1 day to about 24 weeks. As stated above, for the purposes of the present invention, planned regular drug holidays can be incorporated into an administration period without destroying its continuous nature.

The relationship between the total NO-donor content of a patch and the amount of NO-donor that is actually delivered to the skin depends in large part on the adhesive and other materials used in the patch. This relationship is discussed in, for example, U.S. Pat. Nos. 4,954,344; 4,849,226; 4,812,313; and 5,186,938, which to the extent compatible with the teachings of the present invention are incorporated herein by reference.

In addition to the NO-donor, the NO-donor composition of the present invention can also include various binders and excipients as are well known in the transdermal patch arts. Examples include, but are not limited to solvents, permeation enhancers, crosslinkers, and other active compounds, such as nonsteroidal anti-inflammatory compounds (NSAIDS), corticosteroids, salicylic acid and its derivatives, opioids, vasoconstrictors, vanilloid receptors-1 activators, local anesthetics, and menthol. Examples of permeation enhancers include but are not limited to polyethylene glycols, surfactants, and combinations thereof.

The transdermal patches of the present invention can take a wide variety of structural forms, including reservoir patches and matrix patches. In the broadest sense, all patches include an outer layer (or “backing layer”) that is distal to the skin (except where used to attach to the skin around the periphery of the drug-containing portion of the patch). The backing layer protects the drug-containing portion of the patch from the outside environment. A matrix patch includes a drug-in-adhesive layer that is typically attached to the backing layer and which contacts the skin. In matrix patches, the drug and adhesive can be mixed more or less homogenously, or alternatively, the drug and adhesive can be discretized with one or more “islands” of drug. A reservoir patch typically includes a reservoir of drug where the reservoir is defined by the backing layer and a permeable layer of material that contacts the skin and allows the drug to pass therethrough. Both of these types of transdermal patches are well known in the art. In either case, both types of patches have a backing layer which supports, in some way, a NO-donor-containing composition in accordance with embodiments of the present invention.

The backing layer is typically made of plastic or other resilient material and may be impermeable to gas and/or liquid. For patches that are placed on “active” skin regions (e.g., portions of skin that are near or that overlie joints, so that the skin is subject to occasional or frequent stretching or deformation), the backing layer can be formed of a material that is dimensioned and balanced appropriately to meet the need for flexibility (so that the patch does not substantially impede the joint flexing or extending motion) with the need for toughness to resist breakage or other failure.

With specific reference to the types of adhesives that can be present in matrix transdermal patches, or which can be applied to a porous membrane often used for reservoir patches, a wide variety of pharmaceutically-acceptable adhesive polymers can be used in connection with the present invention. Non-limiting examples of adhesives which can be used in the patches of the present invention include acrylic adhesives, polyacrylic adhesive polymers, acrylate copolymers (e.g., polyacrylate), silicone-based adhesives, polyisobutylene adhesive polymers, and combinations thereof. The adhesive matrix can contain varying amounts of the nitric oxide donor depending on the particular donor and the desired dosage and delivery rates. In one embodiment the nitric oxide donor is present in the adhesive matrix in an amount of from 0.1 wt % to 60 wt %. In yet another embodiment the nitric oxide donor is present in an amount from 3.0 wt % to 35 wt %. In another embodiment, the nitric oxide donor is nitroglycerin and is used in the formulation of a transdermal matrix patch. In one embodiment, the nitroglycerin comprises from about 6.0 wt % to about 18.0 wt % of the matrix in the transdermal matrix patch. In yet a further embodiment, nitroglycerin comprises from about 8.0 wt % to about 16.0 wt % of the matrix in the transdermal matrix patch.

### EXAMPLES

The following examples illustrate exemplary embodiments of the invention. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what is presently deemed to be practical embodiments of the invention.

### Examples 1-7

Several prototype nitroglycerin patch formulations are prepared in accordance with embodiments of the present invention with the starting components listed in Table 1.

TABLE 1

| | Nitroglycerin | Ethyl Acetate | DuroTak 87-2194 (liquid) |
|---|---|---|---|
| Example | (Wt %) | (Wt %) | (Wt %) |
| 1 | 2.6% | 7.7% | 89.8% |
| 2 | 3.4% | 10.1% | 86.6% |
| 3 | 3.8% | 11.4% | 84.8% |
| 4 | 4.2% | 12.4% | 83.4% |
| 5 | 4.9% | 14.7% | 80.4% |
| 6 | 5.9% | 17.6% | 76.5% |
| 7 | 6.3% | 18.6% | 75.1% |

The patches of Examples 1-6 are prepared in the following manner:

1. Nitroglycerin is diluted in the DuroTak 87-2194 adhesive and ethyl acetate solvent forming the drug solution. (see Table 1 for examples of dilution ratios)

2. The drug solution of the adhesive blend is formed onto a release liner using a mechanical coater.

3. The coated release liner is then passed through an oven which causes the solvent (e.g. ethyl acetate and the solvent present in the liquid DuroTak) to evaporate, forming a solid, tacky layer of adhesive matrix that contains nitroglycerin dispersed in an adhesive matrix.

4. A polyethylene film is then laminated to the adhesive matrix.

5. The nitroglycerin patch laminate is then cut to specified dimensions using a die cutter and the patches are then individually pouched in sealed foil-lined material.

The resulting patches from Examples 1-7 have finished product content (Wt %) as shown in Table 2

TABLE 2

| | | | Total | Durotack 87-2194 |
|---|---|---|---|---|
| | Dose | Nitroglycerin | Nitroglycerin | (remaining solids) |
| Example | (mcg/hr)* | (Wt %) | (mg/cm²) | (Wt %) |
| 1 | 17.1 | 6.0% | 0.493 | 94% |
| 2 | 23.3 | 8.0% | 0.657 | 92% |
| 3 | 29.2 | 9.1% | 0.75 | 90.9% |
| 4 | 33.3 | 10.0% | 0.82 | 90.0% |
| 5 | 42.9 | 12.0% | 0.985 | 88% |
| 6 | 54.2 | 14.7% | 1.2 | 85.3% |
| 7 | 58.3 | 15.6% | 1.28 | 84.4% |

*Based on a drug delivery zone size of 7.2 cm²

### Example 8

A nitroglycerin containing transdermal matrix patch formulated to deliver 17.1 mcg/hr is applied to a skin site proximate an afflicted Achilles tendon of a human subject experiencing pain affiliated with tendinopathy. The patch has a drug delivery zone of about 7.2 cm². After 24 hours, the patch is removed and replaced with an identical patch. The patch is replaced once daily for a period of two weeks at which time the pain associated with the tendinopathy is reduced.

### Example 9

Same as Example 8, except that the administration period is for 8 weeks.

### Example 10

Same as Example 8, except that the patch is removed after the 12 hour administration period and the subject does not reapply a second patch until after the occurrence of a 12 hour drug holiday period. After the drug holiday, a new patch is applied. This administration period is continued for a period of 6 weeks.

### Example 11

A nitroglycerin containing transdermal matrix patch formulated to deliver 20 μg/hour is applied to a skin site proximate an afflicted shoulder tendon of a subject. The patch has a drug delivery zone of about 100 cm². Although the formulation for the exemplified patch is not listed in Table 1 above, one skilled in the art would be able to deduce the formula based on the information disclosed herein. After an administration period of about 24 hours the patch is removed and a new patch applied in its place. This is repeated daily for a period of 4 weeks at which time the subject has reduced pain and tenderness associated with the afflicted tendon and improved function thereof.

### Example 12

A nitroglycerin containing transdermal matrix patch formulated to deliver 58.3 μg/hour is applied to a skin site proximate to an afflicted elbow of a human subject experiencing pain affiliated with overuse extensor tendinopathy. The patch has a drug delivery zone of 7.2 cm² and the concentration of nitroglycerin in the patch is about 1.28 mg/cm². After 24 hours, the patch is removed and a new patch is applied to a new skin site proximate the afflicted elbow. This pattern is repeated daily for a period of 24 weeks at which time the pain and tenderness associated with the tendinopathy is reduced.

### Example 13

Same as Example 12, except that the initial patch is removed after a period of 12 hours at which time a drug holiday period of 12 hours is allowed to pass and then a second patch is applied to or proximate the same skin site and left for a period of 12 hours. This is repeated for a period of 12 weeks or until the elbow is pain free.

### Example 14

Same as Example except the patch is formulated to deliver 17.1 μg/hour of nitroglycerin for a period of 24 hours.

### Example 15

A nitroglycerin containing matrix patch formulated to deliver about 23.3 μg/hour over a period of 24 hours. The patch has a drug delivery zone of 7.2 cm² and the concentration of nitroglycerin in the patch is about 657 mcg/cm². The patch is applied to a skin site proximate a strained hamstring muscle of a human subject. After 24 hours, the patch is removed and replaced with a second similar patch. This cycle is repeated every 24 hours for 2 weeks or until the muscle is at least substantially healed.

### Example 16

A nitric oxide donor-containing patch is formulated to deliver about 42.9 μg/hour of nitric oxide donor to a subject. Specifically, the nitric oxide donor is nitroglycerin. The patch is applied to a skin site proximate a surgically repaired Achilles tendon of a subject. The patch has a drug delivery zone of 20 cm². The patch is left on the skin site for a period of 24 hours, at which time it is replaced with a similar patch for another period of 24 hours. After 5 weeks of consecutive wearing of the patches, the subject experiences less pain associated with the surgically repaired tendon and has improved function thereof.

### Example 17

A nitroglycerin containing patch is formulated to deliver about 29.1 mcg/hour of nitroglycerin to a subject. The patch is applied to a skin site proximate an area of acute inflammation. The patch has a drug delivery zone of 2.5 cm². The patch is left on the skin site for a period of 48 hours, at which time it is replaced with a new patch. This process is repeated for a period of 2 weeks after which the acute inflammation is reduced.

### Example 18

A nitric oxide donor containing patch is formulated to deliver about 50 mcg/hr of nitric oxide to a patient suffering from tendinosis. The nitric oxide donor is isosorbide dinitrate. The patch has a drug delivery zone of approximately 10 cm². The patch is applied to a skin site proximate an injured elbow tendon a human subject. After 24 hours, the patch is removed and replaced with a second similar patch. This cycle is repeated every 24 hours for 7 weeks or until the subject has reduced pain associated with the injured tendon.

### Example 19

Same as Example 18 except that the nitric oxide donor is isosorbide mononitrate.

### Example 20

A nitroglycerin containing patch is formulated to deliver about 33.3 mcg/hour of nitroglycerin to a subject for a period of 4 hours. The patch is applied to a skin site proximate a surgically repaired wound having associated inflammation and pain. The patch has a drug delivery zone of 2.5 cm². The patch is left on the skin site for a period of 4 hours, at which time it is removed and replaced with a new patch. This process is repeated for a period of 8 weeks after which the inflammation and pain associated with the surgically repaired wound are reduced.

### Example 21

Drug delivery rates were determined for nitroglycerin patches formulated according to Examples 3 and 7. The resulting experimental data is displayed in Table 3.

TABLE 3

| | GTN | GTN Delivered (mg/cm²*) | | | | | | | |
|---|---|---|---|---|---|---|---|---|---|
| Ex- | (wt | 0 | 2 | 4.5 | 8.5 | 24.5 | 29 | 48 | 72 |
| ample | %) | hours | hours | hours | hours | hours | hours | hours | hours |
| 3 | 3.8 | 0 | 0.08 | 0.22 | 0.39 | 0.97 | 1.15 | 1.85 | 2.58 |
| 7 | 6.3 | 0 | 0.16 | 0.32 | 0.55 | 1.62 | 2.04 | 3.16 | 4.36 |

*Patch size is 7.2 cm²

As shown in the table, the low dose patches of the present invention are capable of linear drug delivery over periods of at least 72 hours.

### Example 22

A nitroglycerin-containing composition for use in the manufacture of nitroglycerin containing transdermal patches is prepared. First, the nitroglycerin is desensitized by dilution with ethyl acetate to a 60% nitroglycerin solution. The dilution is generated in a 300 gallon stainless steel vertical mixer by mixing ethyl acetate, with a minimum temperature of 15.6° C., and nitroglycerin. The nitroglycerin is added to the mixer in two parts and mixed for 10 minutes (acceptable mixing range 10-12 minutes) after each addition of nitroglycerin. Prior to dispensing the solution, the weight of the nitroglycerin is verified for an acceptable range of ±5 lbs. The ethyl acetate/nitroglycerin solution is dispensed into 2.5 gallon (U.S.) polyethylene screw-cap jerricans (increments). The ethyl acetate/nitroglycerin increments are transferred to the final mixing operation. As an in-process control, a sample of the ethyl acetate/nitroglycerin solution is collected and analyzed for nitroglycerin content (method TM-P116B) during dispensing into containers against the target specification of 58.0 to 62.0%.

The ethyl acetate/nitroglycerin solution is then blended with an adhesive. The adhesive resin is slowly added to a 40 gallon stainless steel vertical blender. Additional ethyl acetate is added to the blender to target weight. The blender is started and mixed for sixty to sixty-nine minutes (target sixty minutes) until a uniform blend is achieved. The target composition of final nitroglycerin-containing composition is:

| Nitroglycerin (NG) | 11.5% by weight |
|---|---|
| Ethyl acetate, NF | 34.2% |
| DURO-TAK 87-2194 | 54.3% |
| | 100% |

### Example 23

A batch formulation for transdermal nitroglycerin patches is prepared using the nitroglycerin-containing composition of Example 22 and additional adhesive. Batch formulations for patches having two unique nitroglycerin concentrations are set forth in Table 4.

TABLE 4

| | | |
|---|---|---|
| Components | Function | Amount per 10 kg batch |

3.8% Blend

| | | |
|---|---|---|
| Nitroglycerin-containing | Contains the | 3304.3 g |
| composition of Example 22 | Active Ingredient | |
| DURO-TAK 87-2194 | Adhesive | 6695.7 g |

6.2% Blend

| | | |
|---|---|---|
| nitroglycerin-containing | Contains the | 5391.3 g |
| composition of Example 22 | Active Ingredient | |
| DURO-TAK 87-2194 | Adhesive | 4608.7 g |

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A transdermal patch for the delivery of a nitroglycerin, comprising:
a backing layer, and
a nitroglycerin-containing composition supported at least in part by the backing layer, said transdermal patch having a drug delivery zone defined by the area where the nitroglycerin-containing composition contacts an intact human skin site, said drug delivery zone having an area of at least 2.5 cm², said transdermal patch formulated to deliver nitroglycerin at from about 5 μg/hour to about 70 μg/hour, and wherein the nitroglycerin-containing composition comprises from 6.0 wt % to 18.0 wt % nitroglycerin, with the proviso that the patch contains at least 450 μg/cm² of nitroglycerin.

2. The transdermal patch of claim 1, wherein the drug delivery zone has an area from about 2.5 cm² to 100 cm².

3. The transdermal patch of claim 1, wherein the drug delivery zone has an area from about 3 cm to about 50 cm².

4. The transdermal patch of claim 1, wherein the transdermal patch is an adhesive matrix patch.

5. The transdermal patch of claim 4, wherein the nitroglycerin-containing composition is included in the adhesive matrix.

6. The transdermal patch of claim 5, wherein the adhesive matrix includes an acrylic polymer.

7. The transdermal patch of claim 5, wherein the nitroglycerin is present in the nitroglycerin containing composition in an amount of from 8 wt % to 16 wt %.

8. The transdermal patch of claim 1, wherein the transdermal patch is formulated to deliver the nitroglycerin from the nitroglycerin-containing composition for a period of from 4 hours to 7 days.

9. The transdermal patch of claim 8, wherein the period is from 1 day to 3 days.

10. The transdermal patch of claim 8, wherein the period is from 12 hours to 24 hours.

11. The transdermal patch of claim 1, wherein the transdermal patch includes a permeation enhancer.

12. The transdermal patch of claim 11, where in the permeation enhancer is selected from the group consisting of polyethylene glycols, surfactants, and combinations thereof.

13. The transdermal patch of claim 1, wherein the transdermal patch is a reservoir patch.

14. The transdermal patch of claim 13, wherein the transdermal patch includes a rate limiting membrane.

15. The transdermal patch of claim 1, wherein the transdermal patch is formulated to deliver 10 μg/hour to 60 μg/hour.

16. The transdermal patch of claim 1, wherein the transdermal patch provides a delivery rate at the drug delivery zone of from about 1 μg/cm²/day to about 600 μg/cm²/day.

17. The transdermal patch of claim 1, wherein the transdermal patch provides a delivery rate at the drug delivery zone of from about 10 μg/cm²/day to about 280 μg/cm²/day.

18. The transdermal patch of claim 1, wherein the transdermal patch provides a delivery rate at the drug delivery zone of from about 50 μg/cm²/day to about 250 μg/cm²/day.

19. A transdermal patch for the delivery of a nitric oxide donor, comprising:
a backing layer, and
a nitric oxide donor-containing composition comprising at least one nitric oxide donor other than nitroglycerin and supported at least in part by the backing layer, said transdermal patch having a drug delivery zone defined by the area where the composition contacts an intact human skin site, said transdermal patch formulated to deliver the nitric oxide donor other than nitroglycerin at from about 5 μg/hour to about 70 μg/hour, and wherein the nitric oxide donor-containing composition comprises from 6.0 wt % to 18.0 wt % of the nitric oxide donor, with the proviso that the patch contains at least 450 μg/cm² of the nitric oxide donor.

20. The transdermal patch of claim 19, wherein the drug delivery zone has an area of at least 2.5 cm².

21. The transdermal patch of claim 19, wherein the drug delivery zone has an area from 2.5 cm² to 100 cm².

22. The transdermal patch of claim 19, wherein the drug delivery zone has an area from 3 cm to 50 cm².

23. The transdermal patch of claim 19, wherein the nitric oxide donor-containing composition includes a nitric oxide donor selected from the group consisting of isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof.

24. The transdermal patch of claim 19, wherein in addition to the nitric oxide donor that is other than nitroglycerin, the nitric oxide donor-containing composition also includes an amount nitroglycerin.

25. The transdermal patch of claim 19, wherein the transdermal patch is an adhesive matrix patch.

26. The transdermal patch of claim 25, wherein the nitric oxide-containing composition is included in the adhesive matrix.

27. The transdermal patch of claim 25, wherein the adhesive matrix includes an acrylic polymer.

28. The transdermal patch of claim 19, wherein the nitric oxide donor containing composition contains from about 8% to about 16% w/w of a nitric oxide donor.

29. The transdermal patch of claim 19, wherein the transdermal patch is formulated to deliver the nitric oxide donor from the nitric oxide donor-containing composition for a period of from 4 hours to 7 days.

30. The transdermal patch of claim 19, wherein the transdermal patch is 10 formulated to deliver the nitric oxide donor for period of 1 day to 3 days.

31. The transdermal patch of claim 19, wherein the period is from 12 hours to 24 hours.

32. The transdermal patch of claim 19, wherein the transdermal patch includes a permeation enhancer.

33. The transdermal patch of claim 32, where in the permeation enhancer is selected from the group consisting of polyethylene glycols, surfactants, and combinations thereof.

34. The transdermal patch of claim 19, wherein the transdermal patch is a reservoir patch.

35. The transdermal patch of claim 34, wherein the transdermal patch includes a rate limiting membrane.

36. The transdermal patch of claim 19, wherein the transdermal patch provides a delivery rate at the drug delivery zone of from about 1 μg/cm²/day to about 600 μg/cm²/day.

37. The transdermal patch of claim 19, wherein the transdermal patch provides a delivery rate at the drug delivery zone of from about 10 μg/cm²/day to about 280 μg/cm²/day.

38. The transdermal patch of claim 19, wherein the transdermal patch provides a delivery rate at the drug delivery zone of from about 50 μg/cm²/day to about 250 μg/cm²/day.

39. A method for delivering a nitric oxide donor to a subject in need thereof, comprising:
applying a nitric oxide donor-containing transdermal patch to a skin surface, said transdermal patch including a nitric oxide donor-containing composition and having a drug delivery zone defined by the area where the nitric oxide donor-containing composition is contacted by intact human skin site, said transdermal patch formulated to deliver from about 5 μg/hour to about 70 μg/hour, and wherein the nitric oxide donor-containing composition comprises from 6.0 wt % to 18.0 wt % nitric oxide donor, with the proviso that the patch contains at least 450 μg/cm² of the nitric oxide donor.

40. The method of claim 39, wherein the drug delivery zone has an area from 2.5 cm² to 100 cm².

41. The method of claim 39, wherein the nitric oxide donor-containing composition includes a nitric oxide donor selected from the group consisting of nitroglycerin, isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof.

42. The method of claim 39, wherein the nitric oxide donor is nitroglycerine.

43. The method of claim 39, wherein the transdermal patch is a matrix patch.

44. The method of claim 43, wherein the nitric oxide-containing composition is included in an adhesive matrix.

45. The method of claim 39, wherein the transdermal patch is formulated to deliver a nitric oxide donor from the nitric oxide donor-containing composition for a period of from 4 hours to 7 days.

46. The transdermal patch of claim 39, wherein the period is from 1 day to 24 days.

47. The transdermal patch of claim 39, wherein the period is from 12 hours to 24 hours.

48. The method of claim 39, wherein the subject in need thereof is experiencing pain, and the transdermal patch is applied for reducing said pain.

49. The method of claim 39, wherein the subject in need thereof has damaged tissue, and the transdermal patch is applied for accelerating healing of the damaged tissue.

50. The method of claim 37, wherein the subject in need thereof has tendinopathy, and the transdermal patch is applied to improve function of an afflicted tendon.

51. The method of claim 50, wherein the tendinopathy is tendinosis.

52. The method of claim 50, wherein the tendinopathy is tendonitis.

53. The method of claim 39, wherein the transdermal patch provides a delivery rate at the drug delivery zone of from about 1 μg/cm²/day to about 600 μg/cm²/day.

54. The method of claim 39, wherein the transdermal patch provides a delivery rate at the drug delivery zone of from about 10 μg/cm²/day to about 280 μg/cm²/day.

55. The method of claim 39, wherein the transdermal patch provides a delivery rate at the drug delivery zone of from about 50 μg/cm²/day to about 250 μg/cm²/day.

56. A transdermal matrix patch for the delivery of nitroglycerin, comprising:
a backing layer, and
a nitroglycerin-containing composition supported at least in part by the backing layer, said transdermal matrix patch having a drug delivery zone defined by the area where the composition contacts an intact human skin site, said transdermal patch formulated to deliver nitroglycerin at from about 5 μg/hour to about 70 μg/hour, and wherein said transdermal patch provides a delivery rate at the drug delivery zone of from about 1 μg/cm²/day to about 280 μg/cm²/day, with the proviso that the patch contains at least 450 μg/cm² of nitroglycerin.

57. The transdermal matrix patch of claim 56, wherein said transdermal matrix patch provides a delivery rate at the drug delivery zone of from about 10 μg/cm²/day to about 280 μg/cm²/day,

58. The transdermal patch of claim 56, wherein the transdermal patch provides a delivery rate at the drug delivery zone of from about 50 μg/cm²/day to about 250 μg/cm²/day.

59. The transdermal matrix patch of claim 56, wherein the nitroglycerin comprises from about 6.0 wt % to about 18.0 wt % of the nitroglycerin-containing composition.

60. The transdermal matrix patch of claim 56, wherein the drug delivery zone has an area from about 2.5 cm² to 100 cm².

61. The transdermal matrix patch of claim 56, wherein the drug delivery zone has an area from about 3 cm to about 50 cm².

62. The transdermal matrix patch of claim 56, wherein the nitroglycerin-containing composition includes an acrylic polymer.

63. The transdermal matrix patch of claim 56, wherein the transdermal matrix patch is formulated to deliver the nitroglycerin from the nitroglycerin-containing composition for a period of from 4 hours to 7 days.

64. The transdermal matrix patch of claim 63, wherein the period is from 1 day to 3 days.

65. The transdermal patch of claim 63, wherein the period is from 12 hours to 24 hours.

66. The transdermal matrix patch of claim 56, wherein the transdermal matrix patch includes a permeation enhancer.

67. The transdermal matrix patch of claim 66, where in the permeation enhancer is selected from the group consisting of polyethylene glycols, surfactants, and combinations thereof.

68. The transdermal matrix patch of claim 56, wherein the transdermal matrix patch is formulated to deliver 10 μg/hour to 60 μg/hour.

69. A transdermal matrix patch for the delivery of a nitric oxide donor, comprising:
a backing layer, and
a nitric oxide donor-containing composition comprising at least one nitric oxide donor other than nitroglycerin supported at least in part by the backing layer, said transdermal matrix patch having a drug delivery zone defined by the area where the composition contacts an intact human skin site, said transdermal patch formulated to deliver the nitric oxide donor at from about 5 μg/hour to about 70 μg/hour, and wherein said transdermal patch provides a delivery rate at the drug delivery zone of from about 1 μg/cm²/day to about 280 μg/cm²/day, with the proviso that the patch contains at least 450 μg/cm² of the nitric oxide donor.

70. The transdermal matrix patch of claim 69, wherein said transdermal matrix patch provides a delivery rate at the drug delivery zone of from about 10 μg/cm²/day to about 280 μg/cm²/day,

71. The transdermal matrix patch of claim 69, wherein the nitric oxide donor comprises from about 6.0 wt % to about 18.0 wt % of the nitric oxide donor-containing composition.

72. The transdermal matrix patch of claim 69, wherein the drug delivery zone has an area from about 2.5 cm² to 100 cm².

73. The transdermal matrix patch of claim 69, wherein the transdermal matrix patch is formulated to deliver the nitroglycerin from the nitric oxide donor-containing composition for a period of from 4 hours to 7 days.

74. The transdermal patch of claim 73, wherein the period is from 1 day to 3 days.

75. The transdermal patch of claim 73, wherein the period is from 12 hours to 24 hours.

76. The transdermal matrix patch of claim 69, wherein the transdermal matrix patch is formulated to deliver 10 μg/hour to 60 μg/hour.

77. The transdermal matrix patch of claim 69, wherein the nitric oxide donor is selected from the group consisting of isosorbide mononitrate, isosorbide dinitrate, s-nitrose-N-acetylpenicillamine, sodium nitroprusside, molsidomine, N-Acetyl-D,L-penicillamine disulfide, 2-(N,N-Diethylamino)-diazenolate-2-oxide, O²-Vinyl-1-(pyrrolidin-1-yl)diazen-1-ium-1,2-diolate, (±)-2-((E)-4-Ethyl-3[(Z)-hydroxyimino]6-methyl-5-nitro-heptenyl)-3-pyridinecarboxamide, S-nitroso-L-glutathione, 2,5-dihydroxy-N-methyl-N-nitrosoaniline, (Z)-1-(N-Methyl-N-[6-(N-methylammoniohexyl)amino])-diazen-1-ium-1,2-diolate, disodium 1-[2-(carboxylato)pyrrolidin-1-yl]diazen-1-ium-1,2-diolate, hydroxydiazenesulfonic acid 1-oxide, and salts and combinations thereof.
